(19) United States
(12) Patent Application Publication
(10) Pub. No.: US 2008/0203080 A1
(43) Pub. Date: Aug. 28, 2008

(54) Patient Warming Blanket

(76) Inventor: Simon S. Fung, Woodbury, MN (US)

Correspondence Address: 3M INNOVATIVE PROPERTIES COMPANY, PO BOX 33427, ST. PAUL, MN, 55133-3427, US

(21) Appl. No.: 11/722,928

(22) PCT Filed: Dec. 23, 2005
(86) PCT No.: PCT/US05/46748

§ 371 Date: Jun. 27, 2007

Related U.S. Application Data
(60) Provisional application No. 60/640,348, filed on Dec. 30, 2004.

(51) Int. Cl. H05B 1/00; H05B 3/34; A61F 7/00

(52) U.S. Cl. 219/212; 219/528; 607/96

(57) ABSTRACT

The present invention provides a patient warming device and method of application that may be used to retain and provide heat to a patient to prevent the onset of hypothermia.

### CROSS REFERENCE TO RELATED APPLICATION

This application claims priority to U.S. Provisional Patent Application No. 60/640,384, filed Dec. 30, 2004.

### BACKGROUND OF THE INVENTION

A patient under general anesthesia will undergo several physiological changes that inhibit the body's normal thermo-regulatory capabilities, such as depression of thermobuffer centers in the hypothalamus, infusion of intravenous fluid and inspiration of dry anesthesia gases during surgery. Moreover, during surgery the body cavity may be exposed. The incidence of hypothermia occurring after surgery has been estimated to be as great as 60% to 90%.

To prevent hypothermia from occurring, it may be necessary to provide heating to a patient during surgery. In one known device, the generalized control of such a patient's temperature is provided by means of a pliable blanket through which a temperature-controlled fluid is circulated. However, most of the temperature exchange between the blanket and the patient takes place only at the points where the blanket contacts the patient's skin. This can result in a high rate of localized thermal activity where the blanket and the patient's skin are in contact.

In another device, the heat transfer mechanism is temperature-controlled air. The air is circulated inside of a flexible bag that covers the patient. However, the structure of this device can be cumbersome to hospital personnel and prevent access to the patient.

Therefore, a need still exists for devices that efficiently and effectively control the body temperature of a patient.

### SUMMARY OF THE INVENTION

The present invention provides a patient warming blanket comprising an insulating layer, a first barrier layer in contact with the insulating layer, a second barrier layer in contact with the first barrier layer to form a barrier pouch; a first porous layer in contact with the first barrier layer, a second porous layer in contact with the first porous layer to form a porous pouch; a thermal composition within the porous pouch; and a thermal buffer layer proximate the porous pouch. The porous pouch is located between the thermal buffer layer and the insulating layer. The thermal composition is activated by opening at least a portion of the barrier pouch to expose the composition to air. The thermal buffer layer can be attached to the second porous layer on the side opposite the thermal composition or it can be attached to the second barrier layer.

In another embodiment, a patient warming blanket is provided comprising an insulating layer; a first barrier layer; a first porous layer in contact with the first barrier layer to form a partially porous pouch wherein the first porous layer is also a thermal buffer layer; a thermal composition within the partially porous pouch; and a second barrier layer in contact with the first barrier layer to form a barrier pouch wherein the thermal composition and the partially porous pouch are enclosed within the barrier pouch. The first porous layer is positioned between the thermal composition and the patient.

In another embodiment, a patient warming blanket is provided comprising an insulating layer; a thermal device comprising a porous pouch containing a thermal composition located proximate the insulating layer and a thermal buffer layer located proximate the porous pouch; and a barrier layer surrounding the porous pouch in a manner sufficient to prevent the activation of the thermal composition until use.

In most embodiments, the insulating layer has a clo value of at least 0.5 based on ASTM method D1518-85. In addition, the porous pouch can comprise one or more compartments. The thermal composition within the porous pouch can be an iron-containing composition.

In most embodiments, the blanket has multiple porous pouches attached to the insulating layer. The porous pouches containing a thermal composition may each individually have a thermal buffer layer and barrier layers to prevent activation of the thermal composition. The multiple porous pouches may also be covered by a single thermal buffer layer and/or barrier layers.

In one embodiment, the barrier layer is the packaging that surrounds the blanket.

In another embodiment, the insulating layer is folded and the composition in the porous pouch is activated by exposure to air when the insulating layer is unfolded.

A patient warming kit comprising a separately packaged insulating layer and thermal devices is also provided.

Various other features and advantages of the present invention should become readily apparent with reference to the following detailed description. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1A illustrates a cross-sectional view of an exemplary embodiment of the present invention;

FIG. 1B shows a cross-sectional view of an exemplary embodiment of the present invention in use;

FIG. 2 illustrates a cross-sectional view of an exemplary embodiment of the present invention FIG. 3 illustrates a cross-sectional view of an exemplary embodiment of the present invention;

FIGS. 4A, 4B and 4C show a perspective view to illustrate the activation of exemplary embodiments according to the present invention;

FIG. 5 shows a cross-sectional view to illustrate the activation of another exemplary embodiment according to the present invention.

For a more complete understanding of the present invention and the advantages thereof, reference is made to the following description taken in conjunction with the accompanying drawings in which like reference numbers indicate like features.

### DETAILED DESCRIPTION OF ILLUSTRATIVE EMBODIMENTS OF THE INVENTION

The present invention provides a patient warming device and method of application that may be used to retain and provide heat to a patient to prevent the onset of hypothermia. A device incorporating concepts of the present invention may be used to controllably provide heat to a patient.

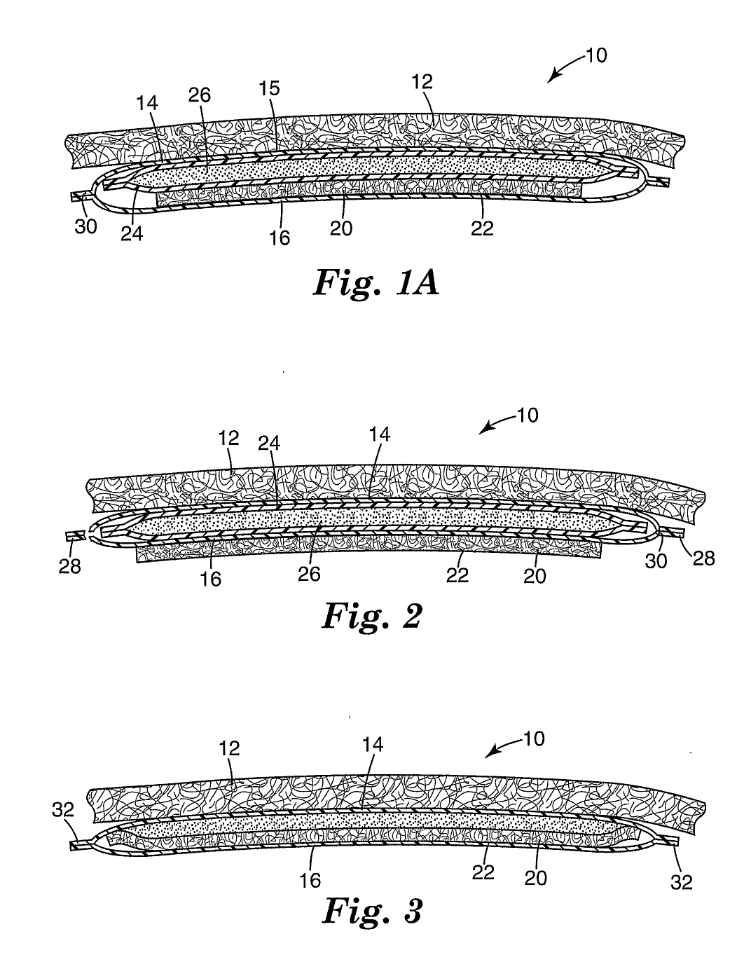

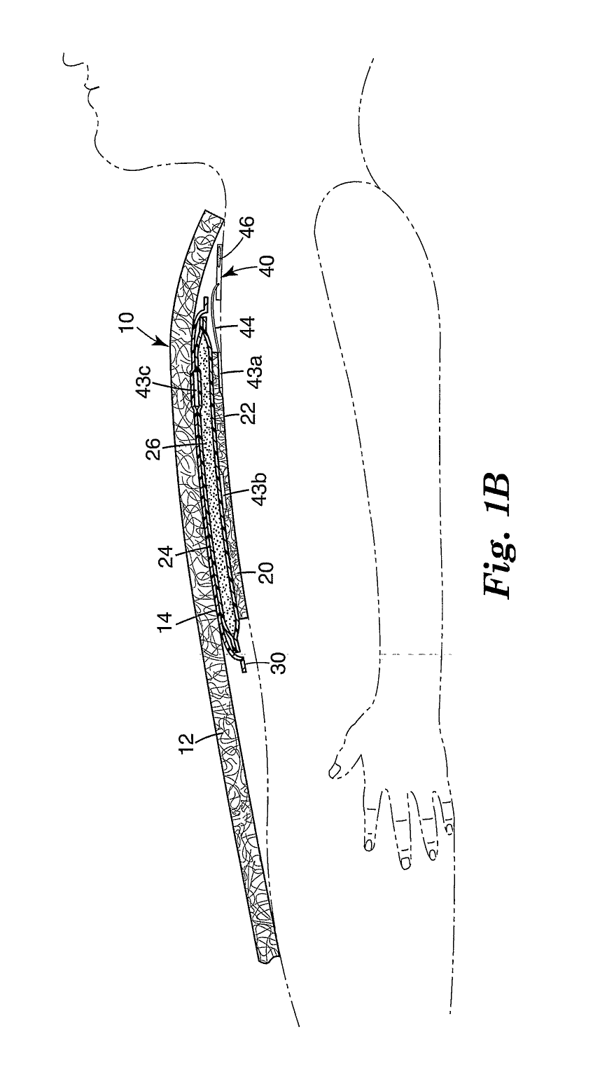

FIG. 1A shows patient warming blanket 10 embodying one concept of the present invention. Blanket 10 includes insulating layer 12, barrier layers 14 and 16, porous pouch 24 containing thermal composition 26, and thermal buffer layer 20.

Insulating layer 12 can be formed from many appropriate materials or combinations of materials, including, but not limited to, nonwoven and woven materials made from polymers such as polyester, polypropylene, polyethylene, polyethylene terephthalate, polyamides, polyvinyl chloride, acrylics, acrylic copolymers, polystyrene, rayons, acetates, and polysulfone. In a preferred embodiment, insulating layer 12 comprises a microfiber-based web made from polypropylene and polyester as described in U.S. Pat. No. 4,118,531 (Hauser) and commercially available from 3M (St. Paul, Minn.) under the brand name THINSULATE.

In most embodiments, insulating layer 12 has a thermal resistance value (clo value) of at least 0.2, more preferably 0.5, and even more preferably 1.0. The do unit is defined as the amount of clothing required by a resting subject to be comfortable at a room temperature of 70° F. (21° C.) using the test method provided in Standard Test Method for Thermal Resistance of Textile Materials, ASTM D1518-85 (Reapproved 2003). Some examples of suitable materials and their respective clo value for use as the insulating layer 12 are given in the table below.

| Material Description | CLO Value |
|---|---|
| 40 grams/sq. m. THINSULATE, C-type | 0.7 |
| 70 grams/sq. m. THINSULATE, C-type | 1.0 |
| 60 grams/sq. m. THINSULATE, G-type | 1.4 |
| 80 grams/sq. m. THINSULATE, G-type | 1.9 |
| 105 grams/sq. m. NEAT SHEET Blanket | 0.5 |
| (polypropylene fibers) available from | |
| Kimberly Clark (Neenah, WI) | |
| 65 grams/sq. m. woven nylon | 0.2 |
| (104 × 104 thread count) | |
| 182.5 grams/sq. m. Cotton Blanket | 0.21 |
| (hospital grade) | |

In a preferred embodiment, porous pouch 24 contains exothermic chemical reactants that form thermal composition 26. Suitable thermal compositions include iron-containing compositions described in U.S. Pat. Nos. 4,649,895; 5,046,479; and 5,918,590. When contacted with air, the thermal composition containing iron reacts exothermically to form iron oxide.

Porous pouch 24 may incorporate other types of thermal heating or cooling devices. Other possible thermal mediums that may be used in porous pouch 24 include, but are not limited to, electrical heating elements, thermoelectric heating or cooling elements, hot or cold liquids, hot or cold gases, and endothermic or exothermic chemical reactants, or combinations thereof.

Porous pouch 24 will preferably be any material permeable to air. For exothermic iron containing compositions, the oxidation reaction depends on the controlled exposure of the iron to air or oxygen. Thus, the flow of air, and the resulting rate of heat generation, will depend on the selection of type of material and its porosity. Suitable materials include polyolefins (e.g., polypropylene, polyethylene), polyurethane, and rayons. In a preferred embodiment, porous pouch 24 is made as described in U.S. Pat. No. 5,046,479 (Usui) with an air permeability of 5000 sec/100 cc.

Referring again to FIG. 1A, blanket 10 includes thermal buffer layer 20 that contacts the patient along skin contact surface 22 when blanket 10 is in use. Thermal buffer layer 20 is typically a nonwoven fabric, but may also be made from a woven material. Suitable materials include polymers such as polyester, polypropylene, polyethylene, polyethylene terephthalate, polyamides, acrylics, acrylic copolymers, polystyrene, rayons, acetates, and polysulfone. Other suitable forms of the polymer include microfiber webs, foam, spunbond and spunlace fabric.

The thickness of the material and its porosity will affect the ability of the thermal buffer layer 20 to maintain the temperature at skin contact surface 22 at or below a target temperature for various patient populations (e.g., children, elderly, physically disabled, etc.). In one embodiment, the temperature of thermal buffer layer 20 is maintained at 43° C. for the duration of the blanket's use. Examples of materials that may be useful include 1.4 oz/sq.yd. spunbond fabric (available from American Nonwoven Corporation, Columbus, Miss.); 40 gram/sq.m. C40 THINSULATE; Spunlace Fabric SX100A 70% Lyocell, 30% Polyester (available from Ahstrom Green Bay Inc., Green Bay, Wis.); Styrofoam packaging material; and 6.5 oz./sq.yd. Struto fabric (available from Struto International, Kings Mountain, N.C.).

Thermal buffer layer 20 and porous pouch 24 may be of substantially identical size as shown in FIG. 1A, or may have different shapes and sizes. If thermal buffer layer 20 extends beyond the edges of barrier layers 14 and 16, it may be preferable to use spunlace or spunbond fabric as the thermal buffer layer 20. The spunbond or spunlace fabric may assist with tear strength and propensity for tearing a straight line in the thermal buffer layer 20 in the machine direction of the fabric. Tearing of the thermal buffer layer 20 may in turn guide the controlled tear of barrier layers 14 and 16.

Thermal buffer layer 20 may be attached to porous pouch 24 by many appropriate means, including, but not limited to, welding or gluing of thermal buffer layer 20, extrusion with porous pouch 24, and adhesion to porous pouch 24.

In the embodiment shown in FIG. 1A, insulating layer 12 and barrier layer 14 are connected along at least a portion of interface 15. Interface 15 may be connected by many appropriate fastening techniques, including, but not limited to, sonic welding, laser welding, adhesive attachment, heat sealing, hook and loop systems, plastic fixtures such as track bars or snaps, and zippers. In addition, interface 15 may be made by the material of insulating layer 12 or barrier layer 14, such as by melting or softening the material. Interface 15 may be made at any desired location or locations, including but not limited to at a point, along a line or edge, or continuously at the interface between insulating layer 12 and barrier layer 14.

Barrier layers 14 and 16 may be made of any suitable material that forms an air impermeable layer. Suitable materials include polyesters (e.g., polyethylene terephthalate, polyethylene naphthalate and polybutylene terephalate and the like), fluorinated layers such as polytetrafluoroethylene (PTFE, e.g., TEFLON), polyamides (e.g., nylon), chlorotriflouroethylene (ACLAR), polyvinylidene fluoride, as well as copolymers of perflourinated monomers with partially fluorinated monomers such as copolymers of tetraflouroethylene/hexafluoropropylene/vinylidene fluoride (THV Fluorothermoplastic from Dyneon Company), polyvinylchloride, polyvinylidene chloride (PVDC, e.g., SARAN HB), ethylene vinyl alcohol (EVOH), and polyolefins (e.g., polyethylene, high density polyethylene, polypropylene, and combinations thereof). A metallic foil film laminate barrier layer, such as aluminum foil, metallized paper or metallized polyester, may also be used.

Barrier layers 14 and 16 are connected at their respective edges to form a seal impermeable to air to prevent premature activation of the thermal composition 26. The sealed edges are provided with perforations 30 to aid in the separation of barrier layers 14 and 16 prior to use. It should be understood that perforations 30 encompasses any means of promoting a guided tear line. The perforations should not be completely through the barrier layer, or activation of the thermal composition could occur. The seal between barrier layers can be provided by any appropriate bonding method, such as adhesive attachment, sonic welding, or melting the material of barrier layers 14 and 16. Alternatively, in other embodiments contemplated within the scope of the present invention, barrier layers 14 and 16 may form one continuous layer with one sealed edge. Regardless of the configuration, the barrier layer functions to maintain an air impermeable layer surrounding the thermal composition 26 prior to use.

FIG. 1B shows the embodiment of FIG. 1A in use according to one aspect of the present invention. In operation, blanket 10 is prepared for use by medical personnel, who remove the blanket 10 from appropriate packaging, if any, and remove the barrier layer 16 by separation of the perforations 30. Removal of the barrier layer 16 activates the heat generating composition 26 in porous pouch 24. Once opened, blanket 10 is then placed on the desired patient surface with thermal buffer layer 20 in contact with the patient along skin contact layer 22. Insulating layer 12 aids in the retention of heat generated by the patient as well as the thermal composition 26.

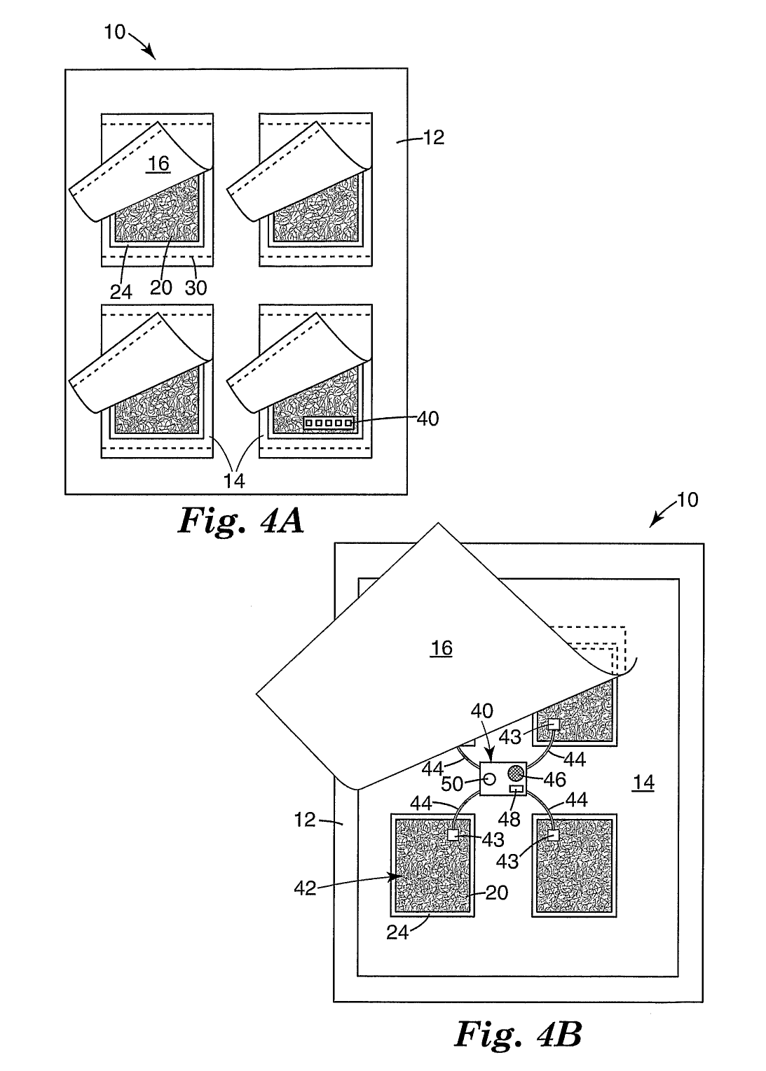

In another embodiment of blanket 10 shown in FIG. 2, thermal buffer layer 20 is attached to barrier layer 16 on the side opposite porous pouch 24. Barrier layers 14 and 16 enclose porous pouch 24 and remain in place during activation. The sealed edges 28 of barrier layers 14 and 16 are removed at perforations 30. Activation of thermal composition 26 occurs through air entering at the perforated ends created by the removal of perforations 30. While thermal buffer layer 20 is shown on the outer surface of barrier layer 16 in FIG. 2, the thermal buffer layer 20 may also be attached to porous pouch 24 and enclosed by barrier layer 16 (as shown in FIG. 1) without removal of the barrier layer 16. In this less preferred embodiment, the skin contact surface 22 (not shown) is on the surface of barrier layer 16 in contact with the skin of the patient.

In an exemplary embodiment shown in FIG. 3, blanket 10 includes insulating layer 12, barrier layers 14 and 16, heat-generating material 26, and thermal buffer layer 20. In this embodiment, the thermal buffer layer 20 also serves as the porous layer to allow the regulated exposure of thermal composition 26 to air after the barrier layer 16 is removed by opening perforations 30.

Blanket 10 in FIG. 3 includes seals 32 that are used to help maintain the impermeability of barrier layers 14 and 16. Barrier layer 14 and 16 can either be folded around porous pouch 24 as shown in FIG. 2 or it may encase porous pouch 24 and thermal buffer layer 20 as shown in FIG. 1. Alternatively, in another embodiment, the barrier layers 14 and 16 may encase the entire blanket 10. The barrier layers 14 and 16 can be hermetically sealed by any appropriate method, including, but not limited to, welding, laser welding, adhesive attachment, and heat sealing. In operation, barrier layers 14 and 16 remain sealed until blanket 10 is used.

Although the embodiments shown in FIGS. 1-3 show perforations 30 as the means for opening barrier layer 14 and/or barrier layer 16, it should be understood that other methods of opening the barrier layer and/or removing the barrier layer are possible. For example, a removable strip of material attached to barrier layer 14 and/or 16 may tear open the barrier layer 14 and/or 16 when removed. In another embodiment, the barrier layer 14 and/or 16 may be embossed to form weakened areas that form a tear line to open the respective barrier layers or that break when the barrier layers 14 and/or 16 are pulled or stretched. In an alternate embodiment, the opening may be accomplished by cutting of the barrier layer 14 and/or 16 with a cutting instrument such as scissors.

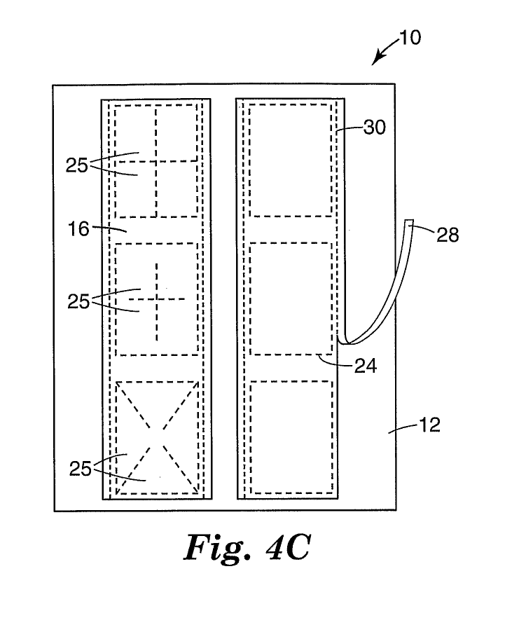

FIGS. 4A, 4B, and 4C illustrate embodiments of the present invention with multiple porous pouches 24 located on insulating layer 12. Although shown in the figures as equidistant from one another, porous pouch 24 could be located at any desired location on blanket 10. Multiple porous pouches 24 allow for more refined distribution of heat from the thermal composition 26 (not shown). Each porous pouch 24 is shown with a thermal buffer layer 20 attached individually. In an alternate embodiment (not shown) thermal buffer layer 20 could cover multiple porous pouches 24 and optionally be co-extensive with the insulating layer 12.

FIGS. 4A, 4B, and 4C also illustrate methods of activating the thermal composition 26. In FIG. 4A, each porous pouch 24 on insulating layer 12 has a barrier layer 16 that can be removed at perforations 30 to activate the blanket 10. This allows the medical professional to select the areas to activate, i.e., in a limited region or over the entire blanket 10, for a given use. In FIG. 4B, multiple porous pouches 24 are covered by a barrier layer 16 that can be removed to activate the multiple porous pouchs 24 all at once. In FIG. 4C, the barrier layer 16 remains over the multiple porous pouchs 24. The sealed edges 28 are removed at perforations 30 to allow air flow to the multiple porous pouchs 24 between barrier layer 16 and barrier layer 14 (not shown).

FIG. 4C also shows porous pouch 24 with multiple compartments 25 in various configurations. While FIG. 4C shows the compartments in fluid communication with each other, multiple compartments 25 may or may not be in fluid communication. In addition, connections may also be used between multiple porous pouches 24 for carrying a fluid (liquid, gaseous or pourable solid) between compartments 25.

In a preferred embodiment of the invention, temperature monitors 40 as shown in FIGS. 1B and 4B can be attached to the porous pouch 24 and/or the thermal buffer layer 20 to provide a reversible temperature indication to observe temperate increases and/or decreases during use. The temperature monitors 40 can be attached at some location on blanket 10 by any suitable means such as adhesives, mechanical fasteners, etc. As used herein, temperature monitors mean a chemical and/or electrical device that monitors the temperature of any heat sources provided for warming patients by detecting and indicating the temperature to a user. The monitor may or may not provide an audible signal or some other form of alerting signal to hospital personnel (i.e., the user) who could then remove or replace the thermal device 42.

The temperature monitor 40 can be a reversible thermometer such as the Reversible Thermometers RLC-70 series available from Omega Engineering, Inc. (Stamford, Conn.). Alternatively, an irreversible temperature indication, particularly for temperature increases can be used such as Non-reversible Omegalabel Temperature Labels commercially available from Omega Engineering, Inc. (Stamford, Conn.).

In a preferred embodiment, the temperature monitor 40 contains a thermistor temperature sensor 43 connected by wires 44 to an electronic module 48 providing circuitry for processing of signal from sensor 43 with an embedded Analog-to-Digital Converter (ADC). The electronic processor 48 would periodically measure the thermistor voltage change, calculate the equivalent resistance, and then reference stored data correlating the temperature to the measured resistance. The temperature monitor 40 preferably has a self-contained power source 50.

If the surface temperature measured by the temperature sensor 43 exceeds a set temperature, the electronic module 48 would trigger an alarm 46 that generates an audible signal. The alarm 46 can be located on the temperature monitor 40 or can be located remote from and in wireless communication with temperature monitor 40. In addition to sound, the alarm 46 can alert the user by other means, including but not limited to visual signal (such as light, colorimetric change) or mechanical signal (such as vibration).

The alarm 46 can be programmed to achieve specific parameters, i.e., the alarm 46 can be programmed shut off after a predetermined period of time, provide a selection of indicators such as visual and/or audible signal, and select different temperature alarm points for various patient populations (e.g., children, elderly, physically disabled, etc.). To maintain the temperature at or below a desired level, the location of the temperature sensor 43 and/or the temperature monitor 40 will affect the desired setting of the alarm 46. In one embodiment, an alarm signal would be triggered once the sensor 43 detected an approximate temperature of 43° C.

One method of activating the temperature monitor 40 includes a tab (not shown) removed from the monitor 40 to allow the temperature monitor 40 to operate. Alternatively, the monitor 40 packaging would be attached to the tab such that the monitor 40 would be activated as soon as the package was opened. Alternatively, the monitor 40 can be activated by means of an on/off switch.

As shown in FIG. 1B, temperature sensor 43a is located at or on the skin contact layer 22. In another embodiment, the temperature sensor 43b is located proximate the interface between thermal buffer layer 20 and porous pouch 24. In a further embodiment, the temperature sensor 43c is located proximate the interface between porous pouch 24 and insulating layer 12. In another embodiment, as shown in FIG. 4B, temperature monitor 40 has one or more temperature sensors 43 positioned and measuring the temperature proximate multiple porous pouches 24. Alternatively, one or more temperature monitors 40 can be used with a single porous pouch 24.

Another feature of a preferred embodiment would include a portion of the insulating layer 12 that allows for marking or identification. Suitable identification methods include a writeable surface such as a smooth film surface or an identification tag.

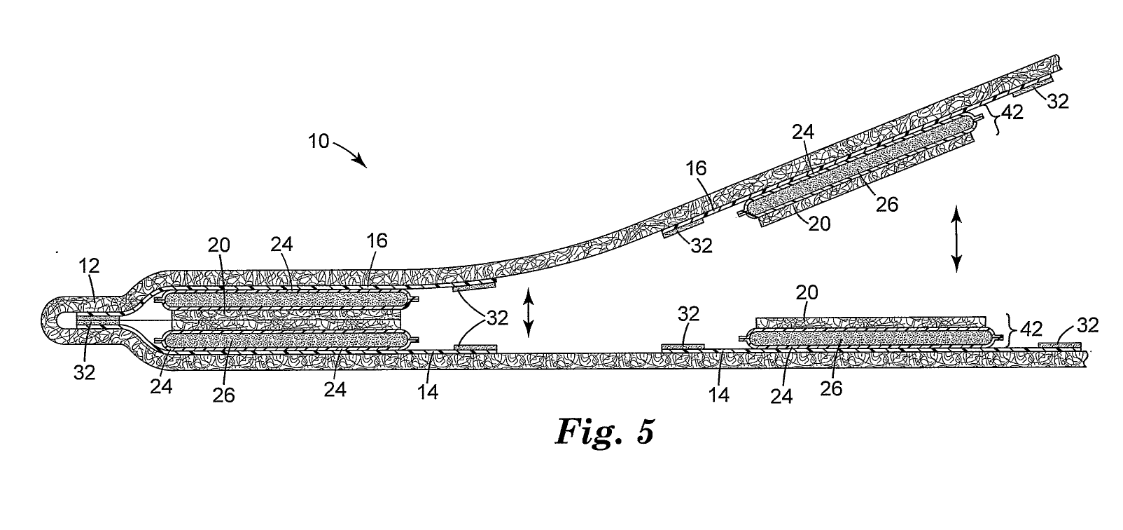

FIG. 5 illustrates unfolding blanket 10 to activate thermal composition 26 according to the teachings of the present invention. Blanket 10 comprises multiple porous pouches 24 with multiple thermal buffer layers 20 to form multiple thermal devices 42. The thermal device 42 comprises at least one porous pouch 24, containing thermal composition 26 and at least one thermal buffering layer 20. Insulating layer 12 is folded to align thermal buffer layers 20 such that it activates at least one thermal device 42 when unfolded. In the embodiment shown in FIG. 5, at least two thermal devices 42 are enclosed between the barrier layers 14 and 16 that are attached to insulating layer 12. Barrier layers 14 and 16 are sealed at their edges to form seals 32. Although shown to enclose at least two thermal devices 42 in FIG. 5, barrier layers 14 and 16 can enclose any number of thermal devices 42 on blanket 10.

In operation, thermal device 42 on blanket 10 is activated by unfolding insulating layer 12. After blanket 10 has been unfolded, it is placed on the patient (not explicitly shown) with thermal buffer layer 20 in contact with the patient.

In an alternate embodiment not shown, multiple thermal devices 42 are attached directly to insulating layer 12. Barrier 14 encloses blanket 10 and serves as packaging for blanket 10. By opening the packaging enclosing blanket 10, thermal composition 26 in thermal device 42 is activated.

While shown in FIG. 1-5 as an assembled blanket 10, an alternate embodiment could provide a patient warming blanket kit that packages the insulating layer separately from the porous pouches with a thermal buffer layer contained in an air impermeable packaging (i.e., the barrier layer). The pouches could be opened and attached to the insulating layer prior to placing the blanket on the patient. In a preferred embodiment, the porous pouches would be adhesively attached to the insulating layer.

Although the present invention has been described in detail, it should be understood that various changes, substitutions, and alterations can be made hereto without departing from the spirit and scope of the invention as defined by the appended claims.

The complete disclosures of the patents, patent documents and publications cited herein are incorporated by reference in their entirety as if each were individually incorporated. In case of conflict, the present specification, including definitions, will control.

Various modifications and alterations to this invention will become apparent to those skilled in the art without departing from the scope and spirit of this invention. It should be understood that this invention is not intended to be unduly limited by the illustrative embodiments set forth herein and that such illustrative embodiments are presented by way of example only with the scope of the invention intended to be limited only by the claims set forth herein as follows.

1. A patient warming blanket comprising:
an insulating layer;
a first barrier layer in contact with the insulating layer;
a second barrier layer in contact with the first barrier layer to form a barrier pouch;
a first porous layer in contact with the first barrier layer;
a second porous layer in contact with the first porous layer to form a porous pouch;
a thermal composition within the porous pouch; and
a thermal buffer layer proximate the porous pouch;
wherein the composition is activated by opening at least a portion of the barrier pouch to expose the composition to air; and
wherein the porous pouch is between the thermal buffer layer and the insulating layer.

2. The blanket of claim 1, wherein the thermal buffer layer is attached to the porous pouch.

3. The blanket of claim 1, wherein the thermal buffer layer is attached to the second barrier layer.

4. The blanket of claim 1, comprising at least two porous pouches with a thermal composition within each porous pouch.

5. (canceled)

6. (canceled)

7. A patient warming blanket comprising:
an insulating layer;
a first barrier layer;
a first porous layer in contact with the first barrier layer to form a partially porous pouch wherein the first porous layer is also a thermal buffer layer;
a thermal composition within the partially porous pouch;
a second barrier layer in contact with the first barrier layer to form a barrier pouch wherein the thermal composition and the partially porous pouch are enclosed within the barrier pouch;
wherein the first porous layer is positioned between the thermal composition and the patient;
wherein the composition is activated by opening at least a portion of the barrier pouch to expose the composition to air.

8. (canceled)

9. (canceled)

10. A patient warming blanket comprising:
an insulating layer;
a thermal device comprising a porous pouch containing a thermal composition located proximate the insulating layer and a thermal buffer layer located proximate the porous pouch; and
a barrier layer surrounding the porous pouch in a manner sufficient to prevent the activation of the thermal composition until use;
wherein the composition in the pouch is activated by opening at least a portion of the barrier layer to expose the composition to air.

11. The blanket of claim 10, wherein the thermal buffer layer is attached to the porous pouch on the side opposite the insulating layer.

12. The blanket of claim 10, wherein the thermal buffer layer is attached to the barrier layer on the side opposite the porous pouch.

13. The blanket of claim 10, wherein the barrier layer further surrounds the thermal buffer layer.

14. (canceled)

15. The blanket of claim 10, wherein the barrier layer is the packaging.

16. (canceled)

17. The blanket of claims 1, 7 or 10, wherein the insulating layer is folded and the composition in the porous pouch is activated by exposure to air when the insulating layer is unfolded.

18. (canceled)

19. The blanket of claims 1, 7 or 10, wherein the barrier layer is releasably attached to the insulating layer.

20. (canceled)

21. (canceled)

22. The blanket of claims 1, 7 or 10, wherein the insulating layer has a clo value of at least 0.5 based on ASTM method D1518-85.

23. A patient warming kit comprising:
an insulating layer with a clo value greater than 0.2; and
a thermal device comprising
a porous pouch comprising a heat generating composition; and
a thermal buffer layer attached to the porous pouch;
wherein the porous pouch can be attached to the insulating layer on the side opposite the thermal buffer layer;
and a barrier layer surrounding the thermal device;
wherein the composition in the compartments is activated by opening the barrier layer to expose the composition to air.

24. The kit of claim 23, wherein the pouch is releasably attached to the insulating layer through any one of adhesive attachment, hook and loop fasteners, plastic fixtures, and zippers.

25. The kit of claim 23, further comprising at least one temperature monitor proximate the thermal buffer layer.

26. The blanket of claims 1, 7 or 10, further comprising at least one temperature monitor proximate the thermal buffer layer.

27. (canceled)

28. A patient warming device comprising:
an insulating layer;
a porous pouch containing a thermal composition;
at least one barrier layer that prevents exposure of the thermal composition to air until activation; and
at least one temperature monitor proximate the porous pouch;
wherein the thermal composition is activated by exposing the composition to air.

29. (canceled)

30. (canceled)

31. A method of warming a patient, comprising
providing at least one porous pouch that contains a heat generating composition;
providing a thermal buffer layer adjacent the patient's skin; and
attaching the at least one porous pouch to the thermal buffer layer.

32. A method of warming a patient, comprising
providing at least one porous pouch that contains a heat generating composition;
providing a temperature monitor proximate the porous pouch; and
positioning the porous pouch proximate the patient's skin.

33. (canceled)

34. (canceled)

35. The method of claim 32, further comprising an insulating layer.

36. (canceled)
